SEQUENCE LISTING (5 sequences)

SEQ ID NO: 1 - DNA, 24 bp, Artificial Sequence
  Forward primer 1 for connecting protein G gene
catatgacgaccgcgtccacctcg

SEQ ID NO: 2 - DNA, 42 bp, Artificial Sequence
  Reverse primer 2 for connecting protein G gene
actgccacctccagtaccgcctccgctttcattatcactgtc

SEQ ID NO: 3 - DNA, 54 bp, Artificial Sequence
  Reverse primer 3 for connecting protein G gene
ggatcctccaccgcttccaccgcctgttccaccgccactgccacctccagtacc

SEQ ID NO: 4 - DNA, 24 bp, Artificial Sequence
  Forward primer 4 for connecting protein G gene
ggatccacttacaaattaatcctt

SEQ ID NO: 5 - DNA, 46 bp, Artificial Sequence
  Reverse primer 5 for connecting protein G gene
ctcgagattagtgatggtgatggtgatgttcagttaccgtaaaggt

(19) United States
(12) Patent Application Publication
(10) Pub. No.: US 2015/0198532 A1
(43) Pub. Date: Jul. 16, 2015

(54) BIOMARKER DETECTING PROBE CAPABLE OF EARLY DETECTION AND PRECISE QUANTIFICATION AND USE THEREOF

(71) Applicant: KOREA INSTITUTE OF SCIENCE AND TECHNOLOGY, Seoul (KR)

(72) Inventors: Kwan Hyi LEE, Seoul (KR); Minhong JEUN, Yangju (KR); Yu Chan KIM, Goyang (KR); Seok LEE, Seoul (KR); Hyun Kwang SEOK, Seoul (KR)

(73) Assignee: KOREA INSTITUTE OF SCIENCE AND TECHNOLOGY, Seoul (KR)

(21) Appl. No.: 14/292,197

(22) Filed: May 30, 2014

(30) Foreign Application Priority Data
Jan. 16, 2014 (KR) 10-2014-0005701

(51) Int. Cl. G01N 21/64; C07K 16/08

(52) U.S. Cl. CPC G01N 21/6428; C07K 16/08; G01N 21/6486; G01N 2021/6439

(57) ABSTRACT

The invention relates to a biomarker detecting probe which is capable of early detection of a biomarker and precise quantification thereof at the same time, and a method of detecting a biomarker using the same. More particularly, it relates to a biomarker detecting probe comprising a ferritin protein, and a targeting antibody linked with a fluorescent material, superparamagnetic nano particle, and conductive particle, and a method of detecting a biomarker using the same.

### CROSS-REFERENCE TO RELATED APPLICATIONS

This application claims the benefit of Korean Patent Application No. 10-2014-0005701, filed on Jan. 16, 2014, in the Korean Intellectual Property Office, the entire disclosure of which is incorporated herein by reference.

### FIELD OF THE INVENTION

This invention relates to a biomarker detecting probe which is capable of early detection of a biomarker and precise quantification thereof at the same time, and a method of detecting a biomarker using the same. More particularly, it relates to a biomarker detecting probe comprising a ferritin protein, and a targeting antibody linked with a fluorescent material, a superparamagnetic nano particle, and a conductive particle, and a method of detecting a biomarker using the same.

### BACKGROUND OF THE INVENTION

A biomarker, which is a type of biomaterials present in biological or medical specimens, functions as a marker capable of diagnosing the condition of a disease by detecting a change in the structure or concentration thereof qualitatively and/or quantitatively and determining the treatment effects of a drug and its correlation with other diseases comprehensively.

The accurate and quick diagnosis of such biomarkers is one of the very important factors in disease management. Particularly, the quick diagnosis is very crucial in early prevention of infectious diseases such as novel influenza, avian influenza, malaria, or dengue. In fact, many people suffered or died globally from the infection of novel influenza or avian influenza occurring in 2009 and this is because these infectious pathogens are highly contagious and cause life threatening symptoms (Dawood F S et al., Emergence of a novel swine-origin influenza A (H1N1) virus in human, N Engl J Med, 360(25), pp 2605-2615, 2009; Beigel J H et al., Avian influenza A (H5N1) infection in human, N Engl J Med, 353(13), pp 1374-1385, 2005). Thus, in order to prevent the pandemic of various infectious diseases in advance, it is essential to accurately determine the results quickly diagnosed at the spot, and for this, it is needed to develop biosensors such as a biomarker detecting probe capable of immediately confirming the presence of a biomarker at the spot. Further, in order to prevent the pandemic of the various infectious diseases in advance, it is needed to build a system capable of transmitting the results quickly diagnosed at the spot to central management system and systematically monitoring them.

For example, hand-foot-mouth disease, which is a viral disease infected via saliva or air by “Coxsackie virus A16” or “Entero virus type 71,” has been rapidly increasing in many Asian countries, and about 1,277,000 patients occurred in China during only the first half of year 2012 and among them, 356 people died, and even in Vietnam, about 63,000 patients occurred and among them, 33 people died (World Health Organization), in Korea, 10.8 out of 1000 patients as of May, 2013 are hand-foot-mouth disease patients, and this is a numerical value increased by 2.25 times in comparison with the same period of the last year. The hand-foot-mouth disease has a latent period of 3 to 7 days and no one can tell the presence of infection until blisters are formed, so that its infection can rapidly spread out during the latent period. Therefore, if it falls under contagious diseases having such latent period, it is even desperate to develop a technology capable of early detecting a biomarker.

For the in-situ diagnosis of biomarkers, a rapid antigen testing has been used, but detection technology about such various contagious diseases has a limit in terms of sensitivity, speed, etc. Specifically, during the novel influenza pandemic in Korea in 2009, there were frequent cases where infected people were reported negative due to the low accuracy of a quick test, leading to the generation of precious victims, who further continued to spread the disease to the surroundings because of the lack of quarantine measures. This evidences the limit of the current quick and accurate diagnosis of biomarkers.

Accordingly, to precisely analyze a biomarker in a quantitative way is required for monitoring infectious or contagious diseases, and for this, methods using ELISA (Enzyme-Linked ImmunoSorbent Assay), and western blotting and mass spectrometry are generally used. In the case of ELISA, polysaccharides or phenol compounds present in test specimens inhibits the reaction, or the concentration of bacteriophages present in the tissues is low, so it is difficult to precisely detect them. The method using mass spectrometry is applicable to analyze a slight amount of biomarkers due to its high sensitivity, but it is usually analyzed through association with a chromatography method so it is difficult to secure repeatability and it has a large analysis data deviation due to machine errors. Further, these methods require excessive labor and huge time.

Moreover, a method for quick diagnosis and a method for quantitative analysis are now separately carried out and consequently, a number of equipment and facilities, and excessive expenses are required.

### SUMMARY OF THE INVENTION

The present invention provides a biomarker detecting probe, comprising a ferritin protein, and a first targeting antibody linked with a fluorescent material, a superparamagnetic nano particle, and a conductive particle which are each independently bound to the ferritin protein, wherein it has magnetic, electrical, and optical properties, it enables a quick and simple early detection at the spot, and it enables precise detection verification and absolute quantification by detecting the magnetic signal of the superparamagnetic nano particle and the electrical signal of the conductive particle.

It is another object of the invention to provide a method of detecting a biomarker, comprising fixing a second targeting antibody specific to a biomarker to be detected onto the inner wall of a vial, injecting a specimen containing the biomarker to be detected to bind the second targeting antibody and the biomarker, mixing a biomarker detecting probe according to the invention with them to sandwich-bind the biomarker detecting probe to the biomarker bound to the second targeting antibody, and detecting a fluorescent material from the targeted probe.

### BRIEF DESCRIPTION OF THE DRAWINGS

FIG. 1 shows an electron microscope photograph of MgₓMn₁₋ₓFe₂O₄ 4-component superparamagnetic ferrite nano particles synthesized through the example.

FIG. 2 is analysis results showing the chemical composition and magnetism properties of MgₓMn₁₋ₓFe₂O₄ 4-component superparamagnetic ferrite nano particle synthesized through the example. “A-site” represents a tetrahedral space of the two spaces of the nano particle, “B-site” represents an octahedral space of the two spaces of the nano particle, and “Ms” represents saturation magnetization. The unit of the “A-site” and “B-site” is at %, and “Bohr,” which is a minimum unit of magnetization per molecule, is used for direct comparison of the magnetization of materials and its unit is uB/molecule.

FIG. 3 shows saturation magnetization and Bohr magneton (Net Moment,_{Ub}) according to the compositions of MgₓMn₁₋ₓFe₂O₄ 4-component superparamagnetic ferrite nano particle synthesized according to Example 1 of the invention.

FIG. 4 illustrates a diagram of the biomarker detecting probe according to one embodiment of the invention where ferritin, an antibody, a fluorescent material and nano particles are linked and an electron microscope photograph thereof.

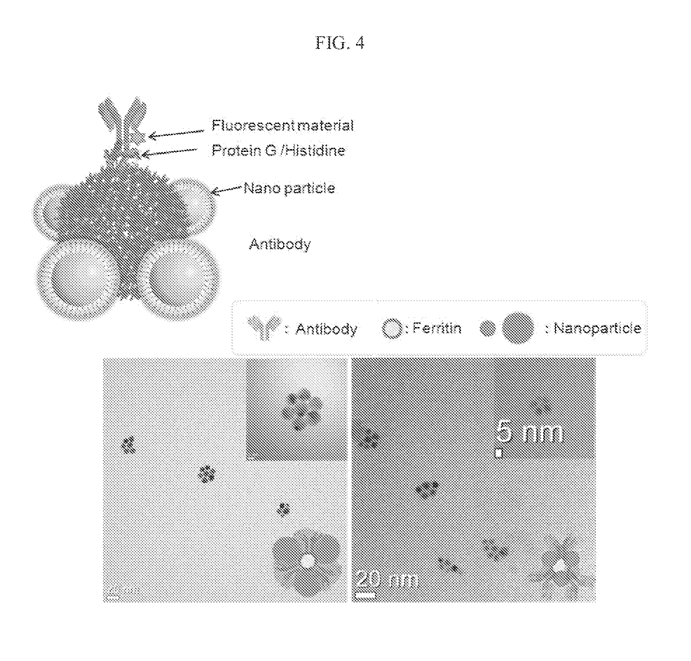

FIG. 5 illustrates a diagram of virus verification system through the detection of a disease biomarker (sandwich-targeting) using the biomarker detecting probe of the invention in a clear vial attached with an antibody and through fluorescence observation using a portable UV lamp, according to one embodiment of the invention.

FIG. 6 illustrates a diagram of a system of detecting the electrical/magnetic signals of gold nano particles and magnetic nano particles of a sensor for detecting the biomarker detecting probe of the invention for the precise confirmation and the absolute quantification of a disease virus.

FIG. 7 illustrates an electron microscope photograph of a sensor for detecting the biomarker detecting probe according to one embodiment of the invention.

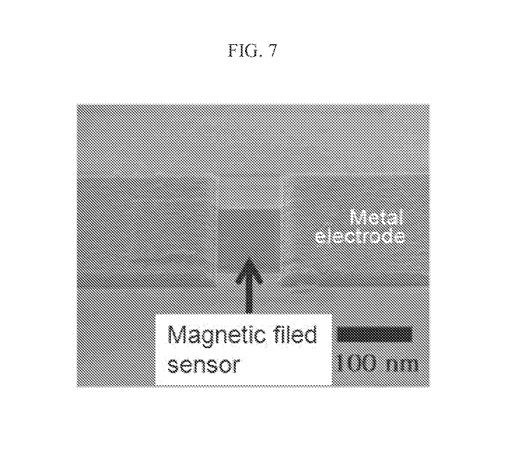

### DETAILED DESCRIPTION OF THE INVENTION

The inventors have confirmed that a biomarker can be early detected in a quick manner by binding an antibody linked with a fluorescent material to a ferritin protein and detecting the fluorescent material, it can be easily separated by binding superparamagnetic nanoparticles to the ferritin protein and applying magnetism, it can be precisely quantitated through a change in magnetic field, and it can be further precisely quantitated by detecting a conductive material bound to the ferritin protein through a change in electrical resistance, and thus completed the subject invention capable of early detection as well as precise quantification of a biomarker.

As one aspect for achieving the above objects, the invention is directed to a biomarker detecting probe, comprising a ferritin protein, and a first targeting antibody linked with a fluorescent material, a superparamagnetic nano particle, and a conductive particle which are each independently bound to the ferritin protein.

As another aspect of the invention, the invention is directed to a method of detecting a biomarker, comprising fixing a second targeting antibody specific to a biomarker to be detected onto the inner wall of a vial, injecting a specimen containing the biomarker to be detected to bind the second targeting antibody and the biomarker, mixing a biomarker detecting probe according to the invention with them to bind the biomarker detecting probe to the biomarker bound to the second targeting antibody, and detecting a fluorescent material from the targeted probe.

The biomarker detecting probe according to the invention is not only capable of detecting a biomarker in a quick and precise manner but also capable of precise quantification. The biomarker detecting probe according to the invention can quickly detect a biomarker by binding a first antibody linked with a fluorescent material, a superparamagnetic nanoparticle and a conductive particle to ferritin respectively so that the first antibody linked with the fluorescent material is targeted at the biomarker and using the fluorescence. Also, the invention uses superparamagnetic particles with no residual magnetization so that the probes do not agglomerate to each other and they only bind to a target biomarker during disease biomarker detection, and they can be easily collected using magnetism separation. Thus, the invention can reduce non-specific reactions and maximize detection efficiency.

The biomarker detecting probe according to the invention can express magnetic, electrical, and optical properties at the same time by binding a superparamagnetic nanoparticle, a conductive particle, and a fluorescence factor to an organic nanoparticle ferritin. Therefore, a quick and simple early detection is possible at the spot which is suspected of the outbreak of a disease by using the optical properties of the fluorescence factor, and a precise detection verification and absolute quantification are possible by detecting the magnetic signal of a superparamagnetic nano particle and the electrical signal of a conductive particle.

The invention will be further described in detail.

The biomarker detecting probe according to one embodiment of the invention comprises a ferritin protein, and a first targeting antibody linked with a fluorescent material, a superparamagnetic nano particle, and a conductive particle which are each independently bound to the ferritin protein.

Throughout the specification, the ferritin protein includes a ferritin protein itself, a heavy chain of ferritin, a light chain of ferritin, an analogue thereof, and apoferritin. Ferritin, which is an assembly of proteins broadly present in extracellular matrix, is composed of 24 single subunits and forms a cage-line nanostructure having the outer diameter of 12 nm and the inner diameter of 8 nm. The ferritin cage has an inside hollow space of about 8 nm, where about 4,500 iron (Fe) atoms are contained at the state of ferric oxide, and it serves to supply such iron atoms during metabolism.

Also, the biomarker detecting probe may further comprise a first linker located between the ferritin protein and the superparamagnetic nano particle, and the linker may be linked at the C-terminal or N-terminal of the ferritin protein and may be a protein or a peptide which does not inhibit the ferritin structure formation of the ferritin protein. More particularly, the ferritin protein and the linker may be prepared as a single fusion protein.

Specifically, the linker may be those used for linking ferritin proteins and superparamagnetic nano particles, and for example, it may be at least one selected from the group consisting of protein G, protein A, protein A/G, Fc receptor, biotin, avidin, histidine, Ni-NTA, or streptavidin.

Throughout the specification, the term “fluorescent material” refers to a material capable of identifying via light that the biomarker detecting probe according to the invention detects a biomarker. For example, it may be at least one selected from the group consisting of Cyanine fluorescent molecule, Rodamine fluorescent molecule, Alexa fluorescent molecule, FITC (fluorescein isothiocyanate) fluorescent molecule, FAM(5-carboxy fluorescein) fluorescent molecule, Texas Red fluorescent molecule and fluorescein, but is not limited thereto and any fluorescent molecules known to an ordinary skilled person fall under the scope of the invention. For instance, it may be selected from quantum dot, tetramethylrhodamine Cy 5, Cy 3, etc.

Through the above fluorescent materials, the presence/absence of a biomarker can be early detected using a simple device such as UV radiation at the spot which is suspected of the outbreak of a disease as well as in laboratory or research center, and this may prevent contagious diseases having a latent period from spreading in advance.

In the invention, the term “superparamagnetic particle” refers to a material with strong magnetism when an external magnetic field is applied. Specifically, it may be a Fe-ferrite doped or substituted with two or more metals selected from the group consisting of Mg, Ni, Gd, Mn, Zn, Dy, and Co. In particular, it may be a 4-component superparamagnetic nano particle, MgₓMn₁₋ₓFe₂O₄which is synthesized by doping or substituting the Fe-ferrite with Mg and Mn.

The superparamagnetic nano particle according to the invention may be a particle having an average diameter of 4 nm to 15 nm. Within the above average diameter ranges, the superparamagnetic nano particle can maintain its superparamagnetism, and if it deviates from the above ranges, superparamagnetism may not be maintained. Further, for instance, the superparamagnetic nano particle may have good separation efficiency when its magnetism is above 60 emu/g but is not limited thereto.

The superparamagnetic nano particle may be a 4-component superparamagnetic nano particle having a high magnetization prepared using a high temperature thermal decomposition method.

In the invention, the “conductive particle” is a particle having excellent electrical conductivity and refers to a particle capable of detection through electrical current between metal electrodes. In other words, any particles that can bring a change in resistance value, current value, etc. by carrying current when power applies thereto may be used as a conductive particle. For example, particles having conductivity of various sizes and materials known to the relevant art such as a gold nano particle, a silver particle, a chrome particle, a clipper particle, an aluminum particle, an iron oxide nano particle, a magnetic bead or a polymer bead containing iron oxide therein may be used as the conductive particle. For example, the average diameter of the conductive particle may be 2 to 15 nm, but is not limited thereto.

In one embodiment of the invention, a first linker may be included between the ferritin protein and the superparamagnetic nano particle, and the first linker may be linked at the C-terminal or N-terminal of the ferritin protein and may be a protein or a peptide which does not inhibit the ferritin structure formation of the ferritin protein. The ferritin protein and the superparamagnetic nano particle may be directly linked, or may be linked using a first linker comprising 30 or less amino acids, preferably 15 to 25 amino acids.

Preferably, the conductive particle may be a gold nano particle. The “gold nano particle” is a particle of gold having a diameter of nanometer size, and its shape and size is not specifically restricted and any shapes and sizes generally used in the pertinent field are appropriate. For example, the gold nano particle may be spherical, and the size of the gold nano particle may be within a range of about 2 nm to about 15 nm on average. The size of the gold nano particle may be suitably defined by the shape of the nano particle, and for example, if the gold nano particle is spherical, its diameter becomes size measure and if the gold nano particle is non-spherical, its size may be defined by the dimension of the longest axis. The gold nano particle used in this invention may be prepared by any ordinary methods used in this technical field, or it may be purchased.

Throughout the specification, the targeting antibody is an antibody having a targeting ability specific to a biomarker to be detected or quantified, and as specific examples, it may be a monoclonal or polyclonal antibody, an immunologically active fragments (e.g., Fab or (Fab)2 fragments), an antibody heavy chain, an antibody light chain, a gene-manipulated single chain molecule, a chimeric antibody, or a humanization antibody, but is not limited thereto and it may be an aptamer, an aptide, or a peptide having targeting ability specific to a biomarker to be detected or quantified.

The biomarker detecting probes according to the invention may detect biomarkers of all kinds of diseases, depending on the types of the targeting antibodies which target biomarkers. In particular, the biomarkers may be biomarkers expressing specifically in bacteria and viruses which are the causes of all kinds of diseases, or may be biomarkers expressing specifically in cells which are the cause of a disease, like cells having different properties from normal cells, such as cancer cells, but are not limited thereto. For example, if a biomarker to be targeted is known and a targeting antibody thereto can be prepared, any biomarkers may fall under the biomarkers of the invention.

The biomarkers detectable by using the detecting probes according to the invention may be biomarkers capable of diagnosing or detecting bacteria such as Anthrax Bacillus, Francisella Turalensis, and Yersinia Pestis; viruses such as Coxsackie A virus, H5N1 virus, enterovirus, foot-and-mouth disease virus, and flu; and cancers such as pancreatic cancer, circulating tumor cells (CTC), prostate cancer, cervical cancer, and liver cancer.

As another aspect, the invention is directed to a method of detecting a biomarker, comprising fixing a second targeting antibody specific to a biomarker to be detected onto the inner wall of a vial, injecting a specimen containing the biomarker to be detected to bind the second targeting antibody and the biomarker, mixing a biomarker detecting probe according to the invention with them to bind the biomarker detecting probe to the biomarker bound to the second targeting antibody, and detecting a fluorescent material from the targeted probe.

In the method of detecting a biomarker according to one embodiment of the invention, the detecting probe and the biomarker to be detected are the same as described above.

As the antibody is fixed onto a vial inner wall, the elimination of the probe which does not bind to the biomarker becomes easy, enabling a quick and accurate detection, in comparison with the case where the antibody is not fixed onto the vial wall and it is injected as a solution state to be reacted with a specimen.

The first targeting antibody and the second targeting antibody may target different portions of the same biomarker. In particular, a portion of the biomarker to be targeted by the first targeting antibody and a portion of the biomarker to be targeted by the second targeting antibody may be portions sufficiently spaced apart so that they do not inhibit their binding with the biomarker.

Also, the method of detecting a biomarker according to the invention may further comprise a step of eliminating a probe which is not bound to the biomarker by applying magnetic force thereto, subsequently to the step of binding the biomarker detecting probe to the biomarker bound to the second targeting antibody. The superparamagnetic particle contained in the probe can be pulled by magnetic three to eliminate the probe which is not bound to the biomarker. The probe which is bound to the biomarker is not eliminated by magnetic force because of its specific binding with the biomarker and the antibody contained in the probe.

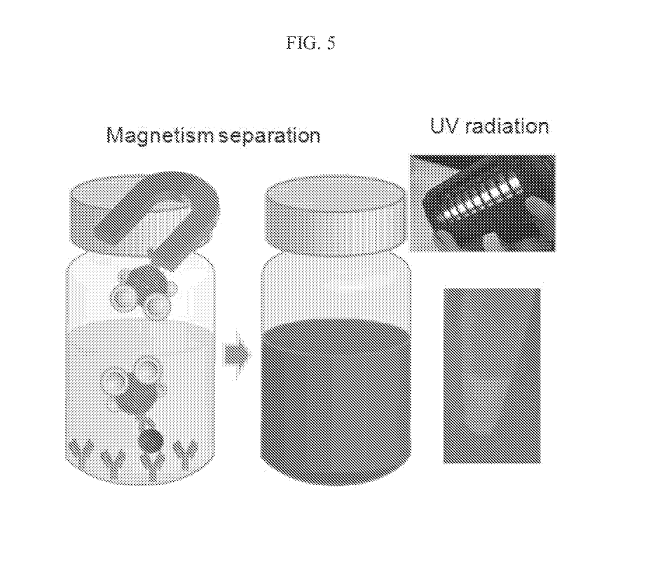

The method of applying magnetic force may be performed by applying magnetic force from the outside of the vial, and for example, magnetic force may be applied by using a magnet, an electromagnet, an alternating current, etc. but any means that can apply magnetic force enough to give attraction force to the superparamagnetic nano particles can be included, without restriction, in the scope of the invention. For instance, as seen in FIG. 5, the biomarker detecting probe which is not bound to the biomarker detected by the second antibody can be separated by applying magnetic force using a magnet outside the vial.

Specifically, the second targeting antibody fixed onto the inner wall of the vial and the first targeting antibody of the biomarker detecting probe according to the invention may sandwich-target the biomarker. Through this, the biomarker can be more accurately detected.

In the method of detecting a biomarker according to the invention, the specimen may be selected from the group consisting of blood, serum, plasma, saliva, urine, excrement, tissue, and cells and it may be obtained directly from a patient who is suspicious of a relevant disease. Also, in case of contagious diseases which spread out via air or water, or spread out via animals such as a mouse or bird, the specimen may be air or water, or it may be blood, serum, plasma, saliva, urine, excrement, tissue and cells from the mouse or bird. Moreover, in case of bacteria or microbes capable of incubation in soils or bacteria or microbes capable of surviving in garbage or faces, the soils or faces may be used as a specimen, but it is not limited thereto. Also, the method of detecting a biomarker may be carried out by further conducting a step of eliminating a probe which is not bound to the biomarker by applying magnetic force, subsequently to the step of binding the biomarker detecting probe to the biomarker bound to the second targeting antibody.

The step of detecting a fluorescent material may be to determine the presence/absence of a biomarker in a specimen by the presence/absence of fluorescence by radiating UV. Hence, the presence/absence of a biomarker may be quickly detected at the spot which is suspected of the outbreak of a disease by simple devices such as a UV lamp. Specifically, the detection of fluorescent materials may be conducted by using a fluorescence detector such as a UV releaser, UV lamp, etc, but any devices which can detect fluorescent materials are included in the scope of the invention. For instance, as seen in FIG. 5, after a biomarker detecting probe which is not bound to the biomarker is eliminated by applying magnetic field outside the vial, the biomarker can be detected by radiating ultraviolet outside the vial to detect fluorescence.

In the present invention, the specimen may be at least one selected from the group consisting of blood, serum, plasma, saliva, urine, excrement, tissue and cells.

As another aspect, the invention is directed to a biomarker detecting kit comprising the biomarker detecting probe of the invention and a detector capable of detecting a fluorescent material.

The second targeting antibody and the first targeting antibody which is included in the biomarker detecting probe according to the invention may be identical or different, and they can be suitably selected by a biomarker to be detected. For example, they may bind simultaneously (sandwich-targeting) to different portions of the same biomarker.

The biomarkers capable of being detected by the biomarker detection method may be particularly biomarkers specifically expressing in a hand-foot-mouse disease causing virus, i.e., Coxsackie virus, an enterovirus, etc. but are not limited thereto, and biomarkers of all kinds of diseases may be detected by the types of the targeting antibodies capable of targeting the biomarkers.

As another aspect of the invention, the invention is directed to a biomarker detecting kit comprising the biomarker detecting probe and a detector capable of detecting a fluorescent material. The detector capable of detecting a fluorescent material is the same as explained in the above.

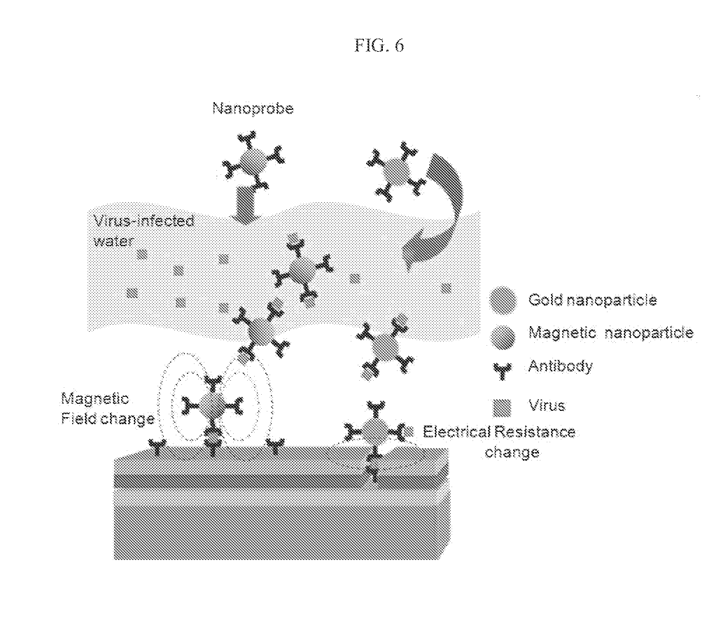

For instance, with reference to FIG. 6 illustrating a biomarker detecting kit, a first antibody of a biomarker detecting probe and a biomarker may be targeted in a sandwich type by fixing a second antibody onto the surface of the kit. The thus targeted biomarker at the surface of the kit in a sandwich type can detect magnetic nano particles linked to the biomarker detecting probe through a change in magnetic field and detect gold nano particles linked to the biomarker detecting probe through a change in electrical resistance, thereby enabling the precise confirmation and the absolute quantification of the biomarker.

This invention relates to a biomarker detecting probe which is capable of early detection of a biomarker and precise quantification thereof at the same time, and by providing technology capable of detecting early at the spot contagious diseases such as a hand-foot-mouse disease virus which is currently impossible of early detection using the developed biomarker detecting probe, it can prevent the diseases from spreading out in advance and contribute to national disease management system by offering accurate information about the diseases through absolute quantification.

Hereafter, the invention will be described in more detail through some examples and comparative examples. However, the following examples are to merely illustrate the present invention, and the scope of the invention is not limited by them in any ways.

### EXAMPLES

### Example 1

### Preparation of a Biomarker Detecting Probe

1-1: Synthesis of Ferritin Linked with Protein G

In order to link the genes for the ferritin protein and Protein G purchased from Promega Co., PCR was carried out using a total of three pairs of primers consisting of five primers. The primers were all purchased from Cosmogenetech (Seoul, Korea), and restriction enzymes Nde I, BamH I, and Xho I were purchased from New England Biolabs (Ipswich, Mass., USA). The primers used are shown in Table 1 below.

TABLE 1

| Designation | Sequence 5′→3′ | SEQ ID NO: |
|---|---|---|
| Forward primer 1 | CATATGACGACCGCGTCCACCTCG | 1 |
| Backward primer 2 | ACTGCCACCTCCAGTACCGCCTCCGCATTTCA | 2 |
| | TTATCACTGTC | |
| Forward primer 1 | CATATGACGACCGCGTCCACCTCG | 1 |
| Backward primer 3 | GGATCCTCCACCGCTTCCACCGCCTGTTCCA | 3 |
| | CCGCCACTGCCACCTCCAGTACC | |
| Forward primer 4 | GGATCCACTTACAAATIAATCCTT | 4 |
| Backward primer 5 | CTCGAGATTAGTGATGGTGATGGTGATGTT | 5 |
| | CAGTTACCGTAAAGGT | |

The ferritin and protein G were linked using a linker of 54 bp, and the linker was divided into two to carry out PCR. First, the ferritin portion was linked to Nde I and linker 1 using primer \textcircled{1} (SEQ ID NO:1) and primer \textcircled{2} (SEQ ID NO:2). Next, primer \textcircled{1} (SEQ ID NO:1) and primer \textcircled{3} (SEQ ID NO:3) were used to link the PCR product (Nde I+Ferritin+Linker 1) and linker 2+BamH I. The Protein G portion was linked to BamH I and Xho I, using primer \textcircled{4} (SEQ ID NO:4) and primer \textcircled{5} (SEQ ID NO:5), and PCR conditions were the same as in Table 2 below. The PCR conditions were set forth in Table 2, and each PCR product was identified by performing Agarose gel electrophoresis of the PCR products.

TABLE 2

| segment | Number of Cycles | Temperature | Time |
|---|---|---|---|
| 1 | 30 | 95° C. | 4 min, 30 sec |
| 2 | 30 | 55° C. | 30 sec |
| 3 | 30 | 72° C. | 40 sec |
| 4 | 1 | 72° C. | 7 min |
| 5 | 1 | 4° C. | ∞ |

The ferritin portion and the Protein G portion produced through the PCR were each treated with restriction enzyme BamH I to cut off end portions from each gene. The two genes with sticky ends were ligated using a ligation enzyme to fuse the ferritin and Protein G.

1-2: Synthesis of 4-Component Superparamagnetic Ferrite Nano Particles

Fe(III) and two kinds of substances selected from the group consisting of Mg, Mn, Ni, Zn, Co, and Gd were added to a 500-mL 3-neck flask containing 20 mL of benzylether and stirred at a room temperature for 20 min. Thereafter, 3.4 mL of oleic acid and 2.6 mL of oleylamine were added to the above produced solution, which was then heated to 200° C. and maintained for 30 min, and then heated again to 296° C. which is the boiling point of benzylether and then maintained for 45 min. Thereafter, the solution was cooled down to a room temperature, rinsed three times with dehydrated ethylalcohol (99.9%) and hexane, and then dried. 4-component superparamagnetic ferrite nano particles collected after dry were carefully ground and kept at a room temperature.

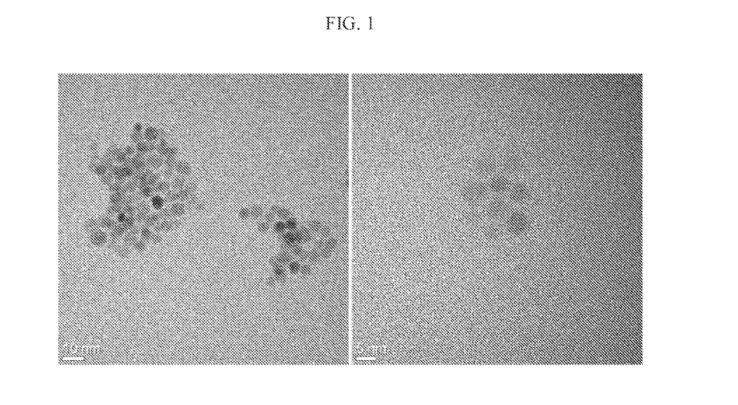

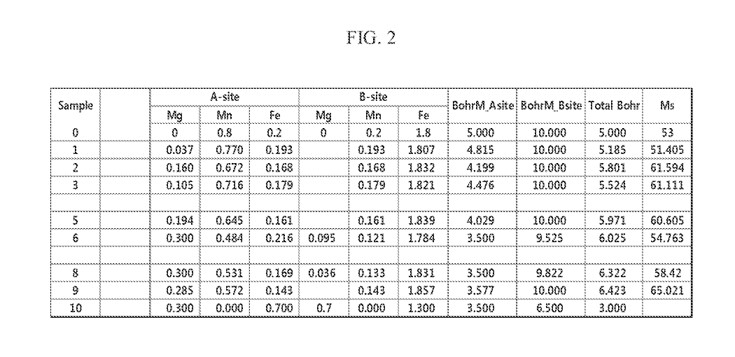

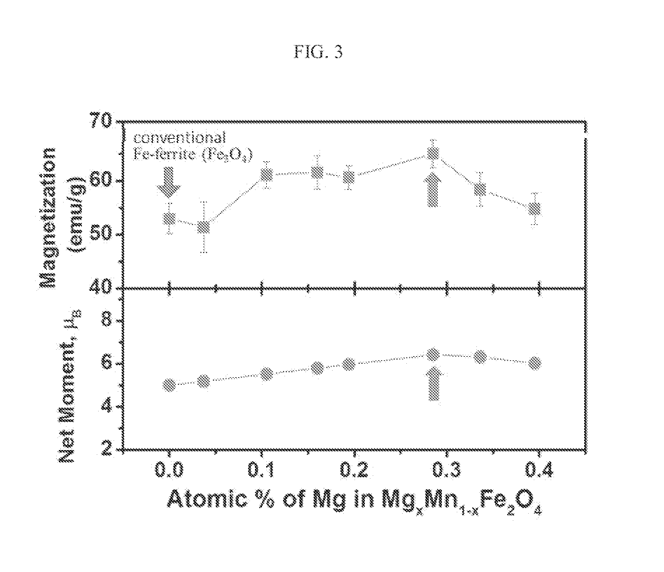

To identify the characteristics of the thus produced superparamagnetic ferrite nano particles, the size of the superparamagnetic ferrite nano particle prepared according to Example 1-2 of the invention was verified using a transmission electron microscope, its magnetization was measured using VSM (vibrating sample magnetometer), and the results are shown in FIG. 1 to FIG. 3.

1-3: Preparation of Biomarker Detecting Probe

The produced 4-component superparamagnetic ferrite nano particles, gold nano particles purchased from Nanoprobe Co., and an antibody linked with fluorescence (R & D systems) were bound to the ferritin prepared in the above proposed method to prepare a biomarker detecting probe. For this, each nano particles were linked with Ni-NTA at their surface according to the following methods. A beaker containing 3.5 mL of D.W. (deionized water) was arranged to float in a sonicator where water temperature was set to 85° C. A combination of lipids (MHPC 270 uL, DPPE-PEG2000 151.2 uL, Ni-NTA 47.7 uL) was added to 70 uL of each nano particles (20 mg/mL) and mixed, and then added dropwise to the beaker inside the sonicator. After sonication for one hour, the solution was centrifuged at 10,000 G for 10 min to collect a supernant only, which was then filtered with a 0.2 um filter and replaced with PBS (phosphate buffered saline, pH 7.4). 10 uL of nano particles linked with Ni-NTA were carefully mixed with 30 ul of 0.5 uM ferritin solution containing ferritin with protein G expressed at its surface and reacted at a room temperature for 1 hour. Histidine present at the surface of the fusion ferritin protein G and Ni-NTA coated onto the nano particles were linked to bind the superparamagnetic nano particles to the surface of the fusion ferritin. The binding of an antibody was carried out by mixing ferritin and 1.8 uL of Hepatitis B virus cortex (HBs) antibody solution having the concentration of 1 mg/ml and then reacting at a room temperature for 1 hour to bind protein G at the surface of the ferritin and an Fc portion of the target antibody. The resultant product was washed three times using a pH 7.4 phosphate buffer solution to eliminate excessive HBs antibodies.

### Example 2

### Virus Detection

The above antibody was attached onto the wall surface of a 20-ml clear vial, which was then filled with 10 mL of phosphate buffer solution. To this solution was added the biomarker detecting probe prepared in Example 1, which was then dispersed. When a sample (saliva, water, etc.) which was suspected of hand-foot-mouth disease virus infection was injected to the above produced solution, the biomarker detecting probe captured the virus, and the virus-captured biomarker detecting probe was bound (sandwich-targeting) to the antibody present on the wall surface of the vial. The biomarker detecting probe which did not capture the virus was eliminated through magnetism separation using a magnet. Lastly, the vial was UV radiated using a portable UV lamp and the color change of the solution was observed to determine the presence/absence of the virus.

As seen in FIG. 5, the presence/absence of the virus was able to be simply determined by examining luminescence when UV was radiated.

### Example 3

### Manufacture of Sensor for Precise Confirmation and Absolute Quantification

A highly precise nano sensor, in which an error for absolute quantification is minimized by simultaneously detecting the magnetic signal and electrical signal of the biomarker detecting probe, was manufactured according to the following methods. A silicon oxide substrate was patterned using photolithography and electron beam lithography and then, an ultra-soft magnetic thin film such as Permalloy was deposited to the thickness of 20 nm using a sputtering system and the pattern was formed thereon using a lift-off method. The above pattern was subject to electrode patterning having a nano gap using an electron beam lithography process, and a gold thin film was then deposited thereto and the pattern was formed thereon using a lift-off method. Lastly, an electrode for contact was patterned using a photolithography process, and a cupper was then deposited thereto to form an electrode. The 4-component superparamagnetic nano particles bound to the biomarker detecting probe are able to be detected by changing the magnetic signal of the ultra-soft magnetic thin film, and the gold nano particles which are located between the gold electrodes of the nano gap are able to be detected by generating a change in electrical conductivity (or resistance). Since the above two signals change in linear proportion to the amounts of the nano particles, the absolute quantification of the captured viruses are possible, and a detection error can be minimized by acquiring and comparing magnetic and electrical signals at the same time.

1. A biomarker detecting probe comprising a ferritin protein, and
a first targeting antibody linked with a fluorescent material, a superparamagnetic nano particle, and a conductive particle which are each independently bound to the ferritin protein.

2. The biomarker detecting probe as claimed in claim 1, further comprising a first linker located between the ferritin protein and the superparamagnetic nano particle, wherein the first linker is linked to the C-terminal or N-terminal of the ferritin protein and it is a protein or a peptide which does not inhibit the ferritin structure formation of the ferritin protein.

3. The biomarker detecting probe as claimed in claim 2, wherein the first linker is at least one selected from the group consisting of protein G, protein A, protein A/G, Fc receptor, biotin, avidin, histidine, Ni-NTA, and streptavidin.

4. The biomarker detecting probe as claimed in claim 2, wherein the ferritin protein and the first linker is constructed as one fusion protein.

5. The biomarker detecting probe as claimed in claim 1, wherein the fluorescent material is at least one selected from the group consisting of Cyanine fluorescent molecule, Rodamine fluorescent molecule, Alexa fluorescent molecule, FITC (fluorescein isothiocyanate) fluorescent molecule, FAM(5-carboxy fluorescein) fluorescent molecule, Texas Red fluorescent molecule and fluorescein.

6. The biomarker detecting probe as claimed in claim 1, wherein the conductive particle has an average diameter of 2 to 15 nm, and it is at least one particle selected from the group consisting of a gold nano particle, a silver particle, a chrome particle, a cupper particle, an aluminum particle, an iron oxide nano particle, a magnetic bead or a polymer bead containing iron oxide therein.

7. The biomarker detecting probe as claimed in claim 1, wherein the superparamagnetic nano particle has an average diameter of 4 nm to 15 nm.

8. The biomarker detecting probe as claimed in claim 1, wherein the superparamagnetic nano particle is a Fe-ferrite doped or substituted with two or more metals selected from the group consisting of Mg, Ni, Gd, Mn, Zn, Dy, and Co.

9. The biomarker detecting probe as claimed in claim 1, wherein the conductive particle and the superparamagnetic nano particle comprise a surface layer containing an amphiphilic substance having a hydrophilic group and a hydrophobic group.

10. The biomarker detecting probe as claimed in claim 9, wherein the amphiphilic substance is at least one selected from the group consisting of 1-Myristoyl-2-Hydroxy-sn-Glycero-3-hosphocholine (MHPC), 1,2-Distearoyl-sn-Glycero-3-Phosphoethanolamine-N-(Methoxy Polyethylene glycol)-2000 (DPPE-PEG2000) and 1,2-dioleoyl-snglycero-3-N-{5-amino-1-carboxypentyl}iminodiacetic acid succinyl nickel salt (Ni-NTA).

11. The biomarker detecting probe as claimed in claim 1, wherein the biomarker is at least one selected from the group consisting of at least one bacteria selected from the group consisting of Anthrax Bacillus, Francisella Turalensis, and Yersinia Pestis;
at least one virus selected from the group consisting of hand-foot-mouth-Coxsackie A virus, enterovirus, H5N1, foot-and-mouth disease and flu; and
at least one cancer selected from the group consisting of pancreatic cancer, circulating tumor cells (CTC), prostate cancer, cervical cancer, and liver cancer.

12. A detection method of a biomarker, comprising fixing a second targeting antibody specific to a biomarker to be detected onto the inner wall of a vial,
injecting a specimen containing the biomarker to be detected to bind the second targeting antibody and the biomarker, and
mixing the biomarker detecting probe according to claim 1 to target the biomarker detecting probe at the biomarker bound to the second targeting antibody, and detecting a fluorescent material from the targeted probe.

13. The detection method as claimed in claim 12, wherein the step of detecting the fluorescent material is to determine the presence/absence of the biomarker in the specimen by examining the presence/absence of fluorescence by UV radiation.

14. The detection method as claimed in claim 12, further comprising a step of eliminating a probe which is not bound to the biomarker by applying magnetic force, subsequently to the step of targeting the biomarker detecting probe at the biomarker bound to the second targeting antibody.

15. The detection method as claimed in claim 12, wherein the specimen is at least one selected from the group consisting of blood, serum, plasma, saliva, urine, excrement, tissue, and cells.

16. The detection method as claimed in claim 12, wherein the biomarker is a biomarker specific to Coxsackie virus, or enterovirus.

17. A biomarker detecting kit comprising the biomarker detecting probe of any one of claim 1 and a detector capable of detecting the fluorescent material of claim 1.

18. A detection method of a biomarker, comprising fixing a second targeting antibody specific to a biomarker to be detected onto the inner wall of a vial,
injecting a specimen containing the biomarker to be detected to bind the second targeting antibody and the biomarker, and
mixing the biomarker detecting probe according to claim 2 to target the biomarker detecting probe at the biomarker bound to the second targeting antibody, and detecting a fluorescent material from the targeted probe.

19. A detection method of a biomarker, comprising fixing a second targeting antibody specific to a biomarker to be detected onto the inner wall of a vial,
injecting a specimen containing the biomarker to be detected to bind the second targeting antibody and the biomarker, and
mixing the biomarker detecting probe according to claim 3 to target the biomarker detecting probe at the biomarker bound to the second targeting antibody, and detecting a fluorescent material from the targeted probe.

20. A detection method of a biomarker, comprising fixing a second targeting antibody specific to a biomarker to be detected onto the inner wall of a vial,
injecting a specimen containing the biomarker to be detected to bind the second targeting antibody and the biomarker, and
mixing the biomarker detecting probe according to claim 4 to target the biomarker detecting probe at the biomarker bound to the second targeting antibody, and detecting a fluorescent material from the targeted probe.
